(19) United States
(12) Patent Application Publication
(10) Pub. No.: US 2006/0083738 A1
(43) Pub. Date: Apr. 20, 2006

(54) Treatment of cancer by the use of anti fas antibody

(75) Inventors: Patrick Gerard Johnston, Belfast (GB); Daniel Longley, Lisburn (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: FUSION ANTIBODIES LIMITED, Belfast (GB)

(21) Appl. No.: 10/514,604

(22) PCT Filed: May 16, 2003
(86) PCT No.: PCT/GB03/02109

§ 371 Date: Dec. 2, 2005

(30) Foreign Application Priority Data
May 17, 2002 (GB) 0211377.7
Jun. 27, 2002 (GB) 0214885.6

(51) Int. Cl. A61K 48/00; A61K 39/395; A61K 31/704; A61K 31/337; A61K 31/28

(52) U.S. Cl. 424/143.1; 514/34; 514/44; 514/449; 514/492

(57) ABSTRACT

The present invention provides a method of killing cancer cells and method of treatment of cancer comprising administration of a therapeutically effective amount of a) a specific binding member which binds to a cell death receptor or a nucleic acid encoding said binding member and (b) a chemotherapeutic agent. The binding member pereferably binds to a Fas receptor. Also described are medicaments for use in treating cancer.

### FIELD OF THE INVENTION

This application relates to a medicament and its use in methods of treatment. In particular, it relates to the treatment of cancer with a death receptor ligand, e.g. a FAS (CD95 or TNF receptor 2) receptor ligand, and a chemotherapeutic agent.

### BACKGROUND TO THE INVENTION

Breast, oesophageal, colorectal, all forms of GI cancer and head and neck cancers are highly malignant with overall 5-year survival rates of less than 50%. The clinical outcome of these patients is predetermined by the presence of widely disseminated tumour cells termed micrometastases with potential for metastatic growth, prior to clinical presentation. Approximately 50% of oesophageal cancer patients are selected for surgical therapy with 30% 5-year survival for this patient sub-group. Randomised clinical trials of neoadjuvant 5FU-based chemotherapy combined with fractionated radiotherapy have demonstrated improvements in survival of 10-20%, although the overall 5-year outcome for the treated groups remains at 30-35%. Those patients who demonstrate complete pathological response in their primary tumours as a result of neoadjuvant treatment have a five-year survival of 80%. Conversely, those patients who do not respond to 5FU-based chemotherapy are denied the opportunity for earlier treatment by surgery or a different neoadjuvant chemotherapeutic based regimen. Thus, there is an urgent need for improved therapeutic strategies.

It is an aim of the present invention to provide an enhanced medicament for treatment of cancers. It is a particular aim to provide a medicament for removal or regression of cell growth of tumour cells.

Fas (CD95/Apo-1) is a member of the TNF cell surface receptor family, which is normally involved in down-regulating the immune response by triggering apoptosis of activated lymphocytes. Binding of Fas Ligand (FasL) causes trimerization of Fas and leads to the recruitment of the adaptor protein FADD (Fas-associated death domain), which in turn recruits procaspase 8 (FADD-like IL-1-converting enzyme, FLICE) to form the death-inducing signalling complex (DISC). Procaspase 8 molecules become activated at the DISC and in turn activate pro-apoptotic downstream molecules such as caspase 3 and the bcl-2 family member BID. This pathway can be inhibited by a number of molecules: c-FLIP (FLICE inhibitory protein) inhibits procaspase 8 recruitment and processing at the DISC; the Fas decoy receptor DcR3 binds to FasL preventing its interaction with Fas; and FAP-1 dephosphorylates Fas thereby inhibiting recruitment of FADD and preventing DISC formation.

### SUMMARY OF THE INVENTION

As described herein, the present inventors have surprisingly shown that by combining treatment using a death receptor ligand, such as an anti FAS antibody, with a chemotherapeutic agent such as 5FU, a synergistic effect is achieved in the killing of cancer cells.

Accordingly, in a first aspect, the present invention provides a method of killing cancer cells comprising administration of a therapeutically effective amount of a) a specific binding member which binds to a cell death receptor or a nucleic acid encoding said binding member and (b) a chemotherapeutic agent.

In a second aspect, the present invention provides a method of treating cancer comprising administration of a therapeutically effective amount of a) a specific binding member which binds to a cell death receptor or a nucleic acid encoding said binding member and (b) a chemotherapeutic agent to a mammal in need thereof.

In a third aspect, there is provided the use of (a) a specific binding member which binds to a cell death receptor or a nucleic acid encoding said binding member and (b) a chemotherapeutic agent in the preparation of a medicament for treating cancer.

In a fourth aspect, there is provided a product comprising a) a specific binding member which binds to a cell death receptor or a nucleic acid encoding said binding member and (b) a chemotherapeutic agent as a combined preparation for the simultaneous, separate or sequential use in the treatment of cancer.

According to a fifth aspect, there is provided a pharmaceutical composition for the treatment of cancer, wherein the composition comprises a) a specific binding member which binds to a cell death receptor or a nucleic acid encoding said binding member and (b) a chemotherapeutic agent and (c) a pharmaceutically acceptable excipient, diluent or carrier.

The invention may be used to treat any cancer. In preferred embodiments of the invention, the cancer is one or more of colorectal, breast, ovarian, cervical, gastric, lung, liver, skin and myeloid (e.g. bone marrow) cancer.

In preferred embodiments of the invention, the binding member is an antibody or a fragment thereof. IN particularly preferred embodiments, the binding member is the FAS antibody CH11 (Yonehara, S., Ishii, A. and Yonehara, M. (1989) J. Exp. Med. 169, 1747-1756) (available commercially e.g. from Upstate Biotechnology, Lake Placid, N.Y.).

The binding member may be bind to any death receptor. Deth receptors include, Fas, TNFR, DR-3, DR-4 and DR-5. In preferred embodiments of the invention, the death receptor is FAS.

In preferred embodiments, the binding member comprises at least one human constant region.

Any suitable chemotherapeutic agent may be used in the present invention. In preferred embodiments, the agent is doxorubicin, oxaliplatin, taxol, tomudex (TDX), 5-Fluorouracil (5-FU), Irinotecan (CPT11) or Cisplatin. Most preferably, the agent is tomudex or 5-Fluorouracil.

The invention also provides a method of treating tumour cells, the method including the steps of administering a compound capable of triggering or binding a death receptor, e.g. a binding member and administering a chemotherapeutic agent. The concentrations of binding members and chemotherapeutic agents used are preferably sufficient to provide a synergistic effect.

The combined medicament thus preferably produces a synergistic effect when used to treat tumour cells.

One aspect of the present invention therefore provides a medicament for use in treating tumour cells, the medicament comprising at least one antibody directed at FAS receptor and at least one cancer chemotherapeutic agent.

The invention also provides a method of treating tumour cells, the method including the steps of administering a compound capable of triggering or binding a death receptor and administering simultaneously, sequentially or separately a chemotherapeutic agent.

In another aspect, the invention provides the use of an antibody directed at FAS receptor in combination with a cancer chemotherapeutic agent in the preparation of a medicament for treatment of tumour cells.

In particular the application relates to the use of an antibody or a fas ligand directed at a death receptor e.g. the FAS receptor (CD95/TNF receptor 2) to synergise with cancer chemotherapeutic agents, e.g. oxaliplatin, 5-FU, and Tomudex, to enhance therapy and enhance the removal or regression of tumour cells.

This application is relevant for, but is not limited to, breast cancer, oesophageal cancer, colorectal cancer, all forms of GI cancer and head and neck cancers and may also be used to target other cells via death receptors.

Preferred features of each aspect of the invention are as for each of the other aspects mutatis mutandis.

### DETAILED DESCRIPTION

Binding Members

In the context of the present invention, a “binding member” is a molecule which has binding specificity for another molecule, in particular a receptor, in particular a death receptor. The binding member may be a member of a pair of specific binding members. The members of a binding pair may be naturally derived or wholly or partially synthetically produced. One member of the pair of molecules may have an area on its surface, which may be a protrusion or a cavity, which specifically binds to and is therefore complementary to a particular spatial and polar organisation of the other member of the pair of molecules. Thus, the members of the pair have the property of binding specifically to each other. Examples of types of binding pairs are antigen-antibody, biotin-avidin, hormone-hormone receptor, receptor-ligand, enzyme-substrate. A binding member of the invention and for use in the invention may be any moiety, for example an antibody or ligand, which can bind to a death receptor.

Antibodies

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd; and diabodies.

The binding member of the invention may be an antibody such as a monoclonal or polyclonal antibody, or a fragment thereof. The constant region of the antibody may be of any class including, but not limited to, human classes IgG, IgA, IgM, IgD and IgE. The antibody may belong to any sub class e.g. IgG1, IgG2, IgG3 and IgG4. IgG1 is preferred.

As antibodies can be modified in a number of ways, the term “antibody” should be construed as covering any binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of such binding fragments are (i) the Fab fragment consisting of VL, VH, CL and CH1 domains;
(ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

A fragment of an antibody or of a polypeptide for use in the present invention generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having death receptor, e.g. FAS neutralisation and/or binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody and the constant/region of a human antibody. Thus the binding member may comprise a human constant region.

The variable region other than the hypervariable region may also be derived from the variable region of a human antibody and/or may also be derived from a monoclonal antibody. In such case, the entire variable region may be derived from murine monoclonal antibody and the antibody is said to be chimerised. Methods for making chimerised antibodies are known in the art. Such methods include, for example, those described in U.S. patents by Boss (Celltech) and by Cabilly (Genentech). See U.S. Pat. Nos. 4,816,397 and 4,816,567, respectively.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

A typical antibody for use in the present invention is a humanised equivalent of CH11 or any chimerised equivalent of an antibody that can bind to the FAS receptor and any alternative antibodies directed at the FAS receptor that have been chimerised and can be use in the treatment of humans. Furthermore, the typical antibody is any antibody that can cross-react with the extracellular portion of the FAS receptor and either bind with high affinity to the FAS receptor, be internalised with the FAS receptor or trigger signalling through the FAS receptor.

Production of Binding Members

The binding members for use in the present invention may be generated wholly or partly by chemical synthesis. The binding members can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a binding member suitable for use in the present invention is to express nucleic acid encoding it, by use of nucleic acid in an expression system. Thus the present invention further provides the use of (a) nucleic acid encoding a specific binding member which binds to a cell death receptor and (b) a chemotherapeutic agent in the preparation of a medicament for treating cancer.

Nucleic acid for use in accordance with the present invention may comprise DNA or RNA and may be wholly or partially synthetic. In a preferred aspect, nucleic acid for use in the invention codes for a binding member of the invention as defined above. The skilled person will be able to determine substitutions, deletions and/or additions to such nucleic acids which will still provide a binding member suitable for use in the present invention.

Nucleic acid sequences encoding a binding member for use with the present invention can be readily prepared by the skilled person using the information and references contained herein and techniques known in the art (for example, see Sambrook, Fritsch and Maniatis, “Molecular Cloning”, A Laboratory Manual, Cold Spring Harbor Laboratory Press, 1989, and Ausubel et al, Short Protocols in Molecular Biology, John Wiley and Sons, 1992), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as construct(s) in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression a specific binding member may be isolated and/or purified using any suitable technique, then used as appropriate.

Binding members-encoding nucleic acid molecules and vectors for use in accordance with the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antibody fragments in prokaryotic cells such as E. coli is well established in the art. For a review, see for example Plückthun, Bio/Technology 9:545-551 (1991).

Expression in eukaryotic cells in culture is also available to those skilled in the art as an option for production of a binding member, see for recent review, for example Reff, Curr. Opinion Biotech. 4:573-576 (1993); Trill et al., Curr. Opinion Biotech. 6:553-560 (1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Sambrook et al., Molecular Cloning: A Laboratory Manual: 2nd Edition, Cold Spring Harbor Laboratory Press (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology, 2nd Edition, John Wiley & Sons (1992).

The nucleic acid may be introduced into a host cell by any suitable means. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells under conditions for expression of the gene.

The nucleic acid may be integrated into the genome (e.g. chromosome) of the host cell. Integration may be promoted by inclusion of sequences which promote recombination with the genome in accordance with standard techniques. The nucleic acid may be on an extra-chromosomal vector within the cell, or otherwise identifiably heterologous or foreign to the cell.

As described above, the present invention is based on the surprising demonstration that combining treatment using a death receptor ligand such as the CH11 antibody with a chemotherapeutic agent results in a surprisingly enhanced synergistic therapeutic effect.

Chemotherapeutic Agents

Any suitable chemotherapeutic agent or agents may be used in the present invention. For example, the agent for use in the invention may include but is not limited to: Doxorubicin, taxol, 5-Fluorouracil (5 FU), Leucovorin, Irinotecan, Mitomycin C, Oxaliplatin, Raltitrexed, Tamoxifen and Cisplatin.

Treatment

Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, gliomas and retinoblastomas.

The compositions and methods of the invention may be particularly useful in the treatment of existing cancer and in the prevention of the recurrence of cancer after initial treatment or surgery.

Administration

Binding members and chemotherapeutic agents may be administered simultaneously, separately or sequentially.

Where administered separately or sequentially, they may be administered within any suitable time period e.g. within 1, 2, 3, 6, 12, 24 or 48 hours of each other. In preferred embodiments, they are administered within 6, preferably within 2, more preferably within 1, most preferably within 20 minutes of each other.

In a preferred embodiment, they are administered as a pharmaceutical composition, which will generally comprise a suitable pharmaceutical excipient, diluent or carrier selected dependent on the intended route of administration.

Binding members and chemotherapeutic agents of and for use in the present invention may be administered to a patient in need of treatment via any suitable route. The precise dose will depend upon a number of factors, including the precise nature of the member (e.g. whole antibody, fragment or diabody) and chemotherapeutic agent.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

It is envisaged that injections (intravenous) will be the primary route for therapeutic administration of compositions although delivery through a catheter or other surgical tubing is also envisaged. Liquid formulations may be utilised after reconstitution from powder formulations.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The binding member, agent, product or composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218,121A; Epstein et al, PNAS USA, 82: 3688-3692, 1985; Hwang et al, PNAS USA, 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

The binding member, agent, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Pharmaceutical Compositions

As described above, the present invention extends to a pharmaceutical composition for the treatment of cancer, the composition comprising a) a specific binding member which binds to a cell death receptor or a nucleic acid encoding said binding member and (b) a chemotherapeutic agent and (c) a pharmaceutically acceptable excipient, diluent or carrier. Pharmaceutical compositions according to the present invention, and for use in accordance with the present invention may comprise, in addition to active ingredients, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

Dose

The binding members, agents, products or compositions are preferably administered to an individual in a “therapeutically effective amount”, this being sufficient to show benefit to the individual. The actual amount administered, and rate and time-course of administration, will depend on the nature and severity of what is being treated. as described herein, the concentrations are preferably sufficient to show a synergistic effect. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration. For example, with respect to binding members, in general, a serum concentration of polypeptides and antibodies that permits saturation of receptors is desirable. A concentration in excess of approximately 0.1 nM is normally sufficient. For example, a dose of 100 mg/m² of antibody provides a serum concentration of approximately 20 nM for approximately eight days.

As a rough guideline, doses of antibodies may be given in amounts of 1 ng/kg-500 mg/kg of patient weight. Equivalent doses of antibody fragments should be used at the same or more frequent intervals in order to maintain a serum level in excess of the concentration that permits saturation of death receptor.

Doses of chemotherapeutic agent will depend on the factors described above but preferably are administered in doses which are within the normal range or, preferably, at a lower concentration than the normal range.

Doses of the binding members may be given at any suitable dose interval e.g. daily, once, twice or thrice weekly.

For example, the periods of administration of a humanised antibody could be from 1 bolus injection to weekly administration for up to one year in combination with chemotherapeutic agents. The likely dose is upwards of 1 mg/per kg/per patient.

It is anticipated that in embodiments of the invention the binding members and chemotherapeutic agent could be given in combination with other forms of chemotherapy or indeed radiotherapy.

The invention provides a combined medicament comprising at least one antibody directed at FAS receptor and at least one cancer chemotherapeutic agent for the synergenic treatment of tumour cells.

The invention will now be described further in the following non-limiting examples. Reference is made to the accompanying drawings in which:

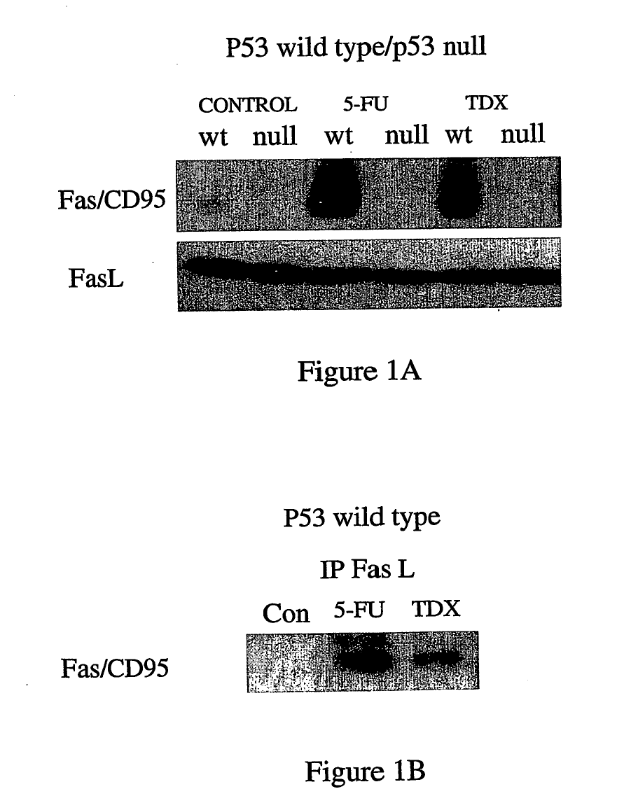

FIG. 1 illustrates FAS/CD95 expression in response to 5-FU and tomudex (TDX).

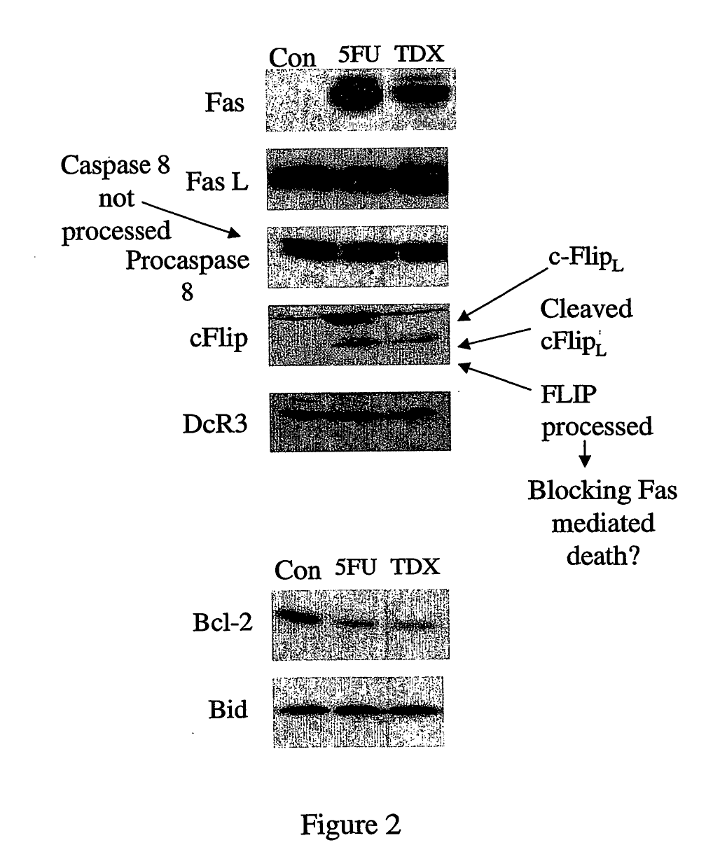

FIG. 2 illustrates expression of apoptosis regulating proteins in MCF-7 cells 72 hours after treatment with 10 μM 5-FU or 100 nM TDX.

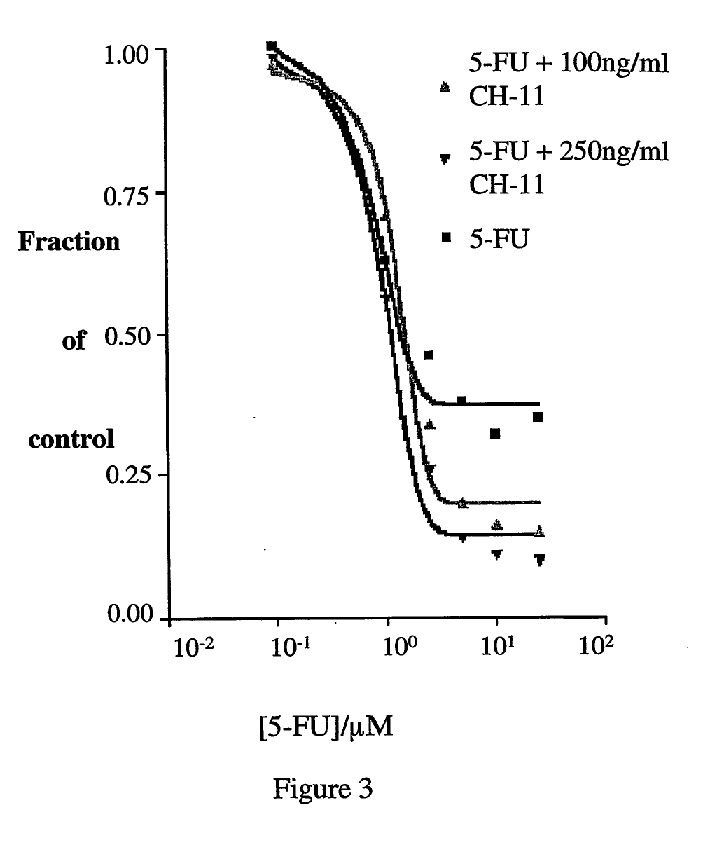

FIG. 3 illustrates MCF-7 response to 72 h pre-treatment with 5FU followed by 24 h CH-11 measured by MTT assay.

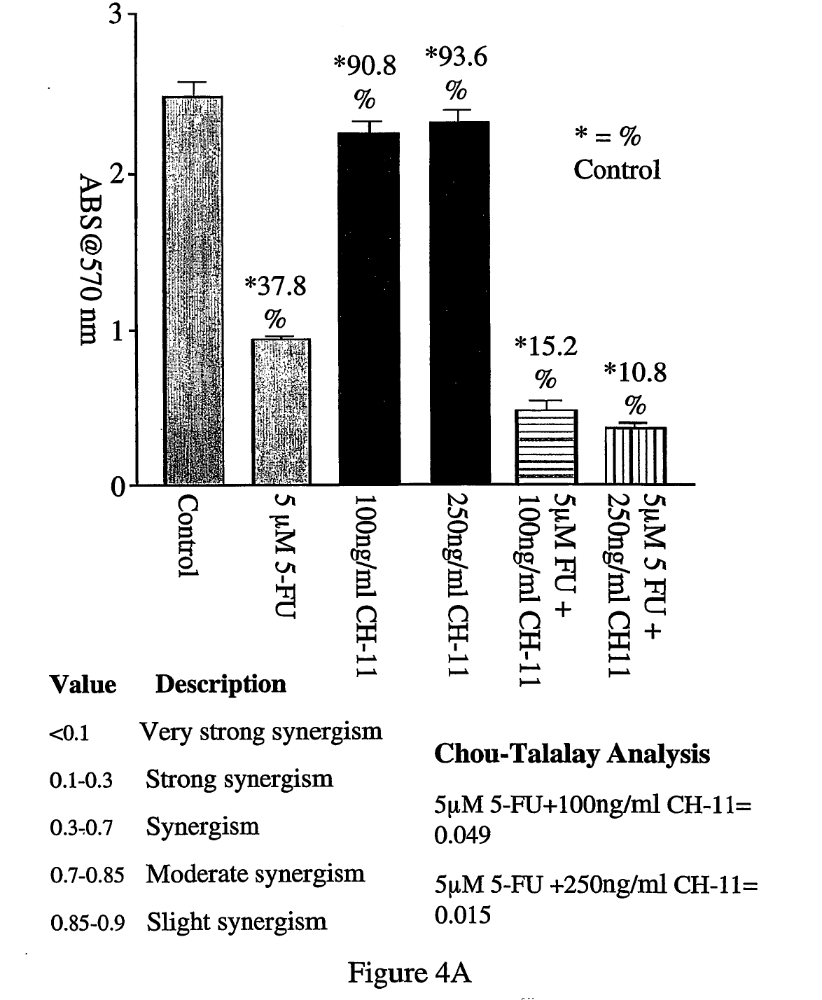

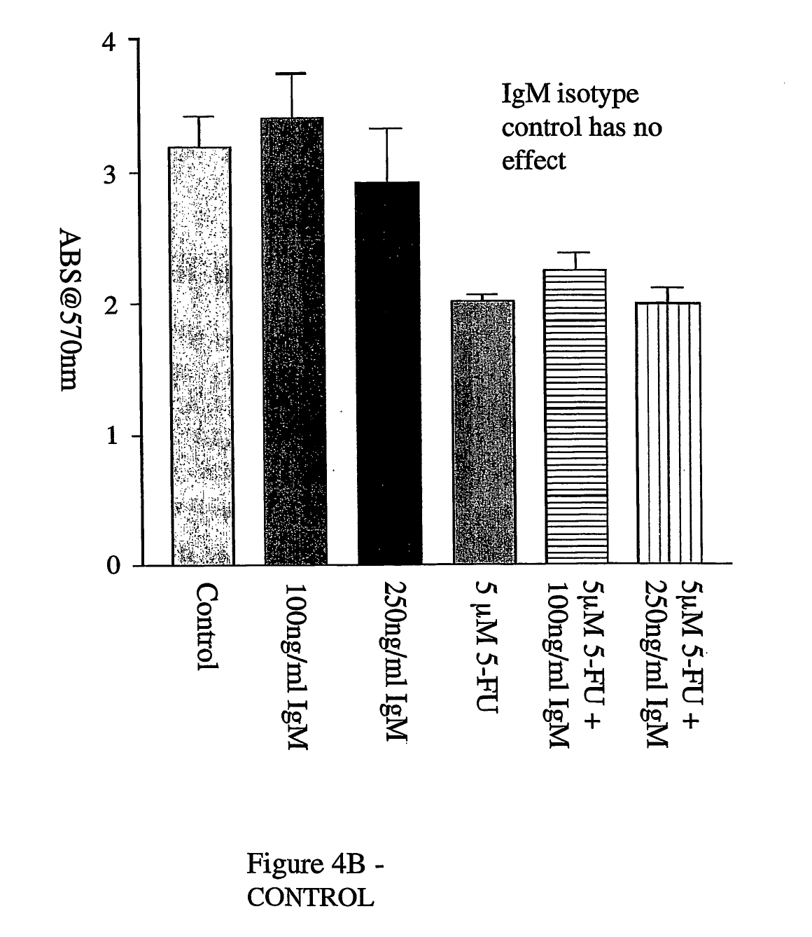

FIG. 4a MCF-7 response to 72 h pre-treatment with 5FU followed by 24 h CH-11 measured by MTT assay.

FIG. 4b illustrates MCF-7 response to 72 h pre-treatment with 5FU followed by 24 h IgM control.

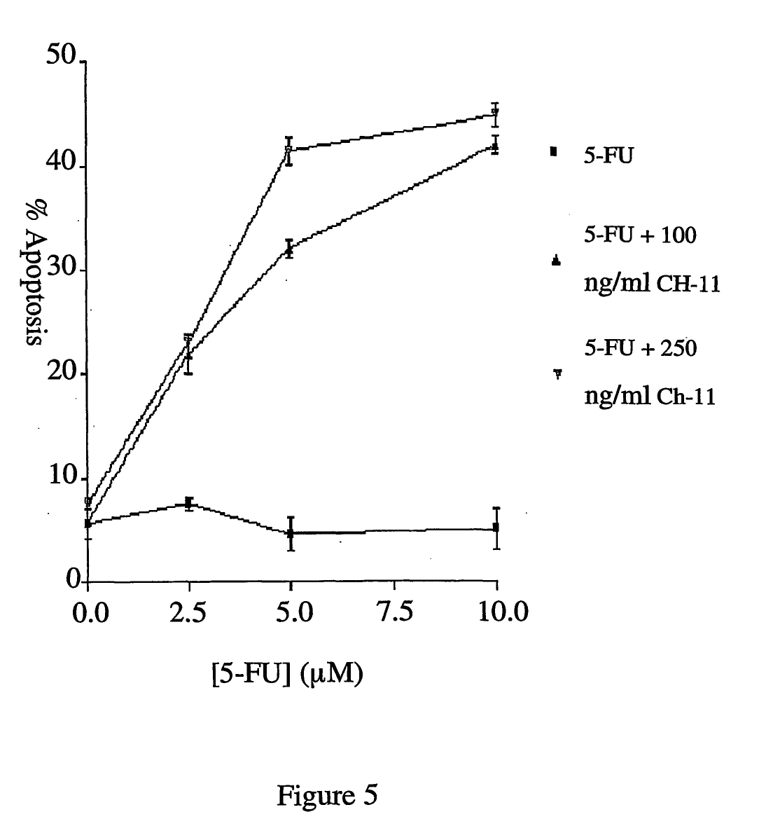

FIG. 5 illustrates Induction of Apoptosis in MCF-7 cells after treatment for 96 h with 5FU+/−CH-11.

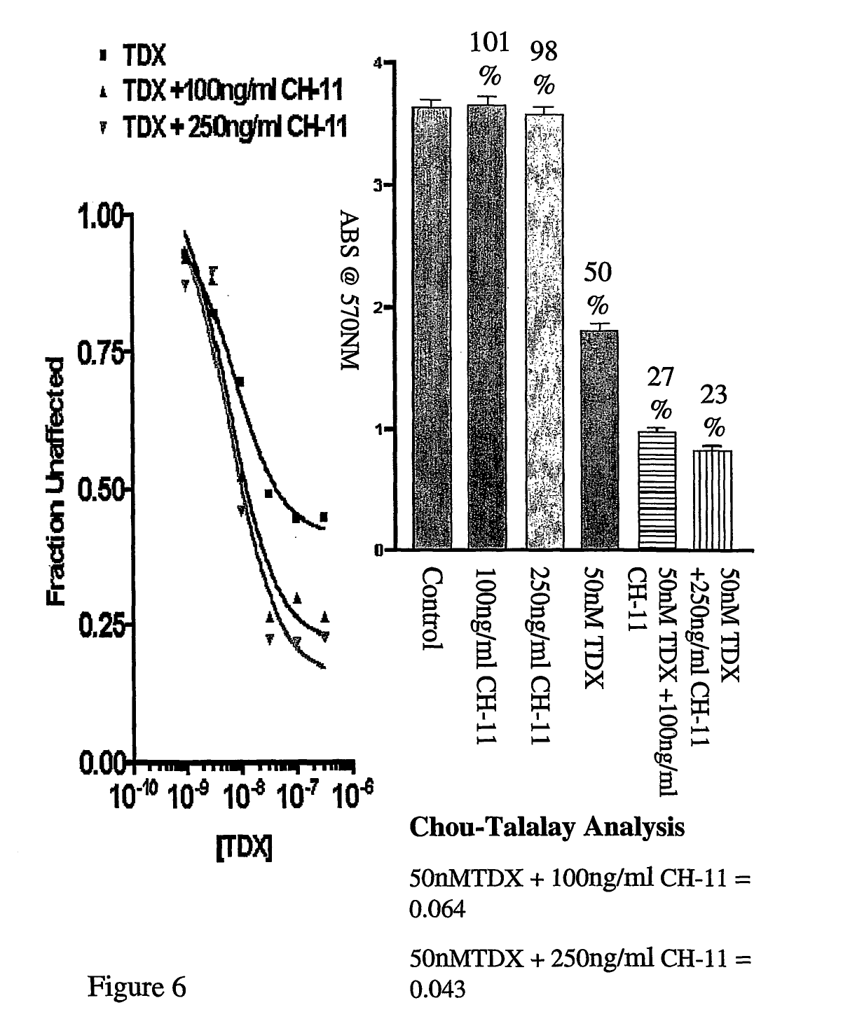

FIG. 6 illustrates synergy between TDX and CH-11.

The data provided shows by Chou Talalay analysis in combination index a strongly significant (p<0.01) synergistic kill of cancer cells using antibody directed at FAS receptor to synergise with cancer chemotherapeutic agents showing that there is very strong synergy in the kill.

The FAS receptor is upregulated following treatment with chemotherapeutic agents and the FAS antibody itself. Increased expression of FAS is induced by co-treatment with CH11 and chemotherapy and anti-FAS antibody and chemotherapy blocks the ability of FLICE to prevent cell death. This is important as it will overcome the inactivation of the caspase pathways as is a common feature of chemotherapy resistant tumours.

The present inventors have also found that FasL is overexpressed in a high percentage of oesophageal cancers. They have developed the concept of the ‘Fas counterattack’ in which tumour cells overexpress FasL to induce Fas-mediated apoptosis of tumour-infiltrating lymphocytes, thereby inhibiting the antitumour immune response. Such a strategy requires that the tumour cell itself is resistant to Fas-mediated cell death. Potential mechanisms of acquired resistance include down-regulation of Fas and up-regulation of Fas inhibitors. Indeed, Fas down-regulation and c-FLIP and DcR3 overexpression have been reported in colon cancers. However, expression of these genes has not as yet been examined in oesophageal tumours, breast cancer, colorectal cancer, or forms of GI cancer and head and neck cancers.

The present inventors found that the expression of Fas is up-regulated >10-fold in the MCF-7 breast cancer and HCT116 colorectal cancer cell lines in response to 5FU treatment. However, this does not result in activation of procaspase 8 or BID (FIG. 1A). Although FasL expression is unaffected by 5FU treatment, immunoprecipitation reactions demonstrate that the interaction between receptor and ligand is up-regulated (FIG. 1B). Analysis of c-FLIP expression in these 5FU-treated cells has demonstrated that its expression is up-regulated and revealed the presence of a truncated form of the protein that is generated during inhibition of procaspase 8 activation at the DISC (FIG. 1A). FACS analysis has revealed that apoptosis of 5FU-treated cells is stimulated by co-treatment with the anti-Fas monoclonal antibody (FIG. 2). Furthermore, MTT cell viability (FIG. 3, 4, 5) and clonogenic survival assays demonstrate a very strong synergistic interaction between 5FU and CH-11. Moreover, procaspase 8 and BID cleavage are not observed in cells treated with either 5PU or anti-Fas monoclonal antibody alone. However, both are activated following co-treatment with 5FU and anti-Fas monoclonal antibody (indicated by loss of full-length procaspase 8 and BID). In addition, c-FLIP expression is not detectable in cells co-treated with 5FU and anti-Fas monoclonal antibody (FIG. 4).

The inventors have observed similar synergistic interactions between anti-Fas monoclonal antibody and both TDX (FIG. 6) and oxaliplatin (data not shown) in MCF-7 and HCT116 cell line models. These results suggest involvement of c-FLIP in blocking Fas-mediated apoptosis following chemotherapeutic treatment e.g. 5FU treatment. Fas-targeted antibodies may thus be used to stimulate apoptosis in chemosensitised cancer cells.

The invention is further verified as described below.

A large group of primary and metastatic oesophageal cancer specimens are collected from patients treated using a neoadjuvant 5FU-based approach, including a complete clinical history.

Micrometastases are present in the bone marrow of resected rib segments in the majority of oesophageal cancer patients. Moreover, the presence of bone marrow micrometastases predicts for early occurrence of metastases. These micrometastatic cells were also found to be present in 13 of 15 patients after neoadjuvant 5FU based therapy, indicating in vivo drug resistance of metastatic cells. The present inventors have found these cells to be viable, to grow in culture, be tumourigenic in nude mice and to possess an angiogenic phenotype. Consistent with the pro-angiogenic function, these cells have been found to express vascular endothelial growth factor (VEGF), basic fibroblast growth factor (bFGF) and a wide spectrum of matrix metalloproteinases involved in invasion and angiogenesis. These observations suggest that bone marrow micrometastases are representative of disseminated metastatic stem cells and may be the appropriate targets for development of effective systemic treatments.

These oesophageal tumour samples from primary and metastatic disease sites along with tumour cell lines provide an important system by which to further define the biological relevance of the Fas cell death pathway in determining response to chemotherapy and outcome in this disease.

Studies are used to define the role of the Fas death receptor pathway in tumours such as primary oesophageal tumours and micrometastatic disease and to determine their value as predictors of clinical outcome and response to 5FU-based therapy. Tumour biopsies are obtained at diagnosis or staging endoscopy before treatment and divided for diagnostic verification and storage in liquid nitrogen prior to analysis. After neoadjuvant 5FU-based therapy, tumour biopsies are obtained from the resected specimen and similarly handled. For analysis of micrometastases, bone marrow samples are obtained from resected rib segments (part of standard thoracotomy). Bone marrow samples are immediately divided into three parts for diagnosis, storage in liquid nitrogen and a specimen for cell line culture. The diagnosis and enumeration of micrometastases is made by immunocytology of cytospins and flow cytometry after staining for cytokeratin 18. The culture of bone marrow cells is performed to obtain micrometastases adherent to coverslips for microdissection and to develop cell lines where possible. These techniques are standard in the Cork Cancer Research Centre.

Fas is evaluated as an effector of apoptosis in cancers such as oesophageal cancers treated with chemotherapeutic agents (5FU, TDX, oxaliplatin and/or CPT-11) using cell lines derived from micrometastatic cells. Initially, Fas, FasL, caspase 8 and BID expression is assessed in the micrometastatic cell lines pre- and post-drug treatment by Northern and Western blot analyses. To define the effect of the Fas signalling pathway on drug-induced cell death, the ability of an anti-FasL monoclonal antibody e.g. from Fusion Antibodies) is evaluated using MTT cell viability and clonogenic survival assays for ability to increase drug resistance. In addition, the effect of anti-Fas antibody on chemotherapy-induced apoptosis is assessed by FACS and TUNEL assays and by analysing caspase 8 and BID activation by Western blot.

As a complementary approach, the effect of the Fas agonistic antibody CH-11 on drug sensitivity is assessed. Specifically, micrometastatic cell cultures are examined for increased drug sensitivity following co-treatment with CH-11 using clonogenic, MTT, FACS and TUNEL assays. In addition, caspase 8 and BID activation are determined in the CH-11 co-treated cultures.

Preclinical development is used to correlate gene expression in the primary tumours from 100 patients with cancers such as oesophageal cancer treated with 5FU-based neoadjuvant therapy and micrometastatic disease from a subset of these patients. Clinical outcome data is test stratified according to several criteria including target gene status, or alternatively treatment responses, and analysed e.g. using the methods of Kaplan and Meier with log rank analysis. The relative contribution of individual pathological and investigative variables is determined e.g. by Cox proportional hazard analysis with backward elimination. Differences between categorical variables is examined e.g. using Fishers exact test.

This can further verify and define the role of Fas-mediated cell death in response to chemotherapy in cancer.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1-8. (canceled)

9. A method of killing cancer cells comprising administering a therapeutically effective amount of a) a specific binding member which binds to a cell death receptor or a nucleic acid encoding said binding member and (b) a chemotherapeutic agent.

10. A method of treating cancer comprising administration of a therapeutically effective amount of a) a specific binding member which binds to a cell death receptor or a nucleic acid encoding said binding member and (b) a chemotherapeutic agent to a mammal in need thereof.

11. The method according to claim 9 or claim 10 wherein the cancer is one or more of colorectal, breast, ovarian, cervical, gastric, lung, liver, skin and myeloid cancer.

12. The method according to claim 9 or 10 wherein the binding member is an antibody or a fragment thereof.

13. The method according to claim 9 or 10 wherein the death receptor is FAS.

14. The method according to claim 9 or 10 wherein the binding member is the anti-FAS antibody CH11.

15. The method according to claim 9 or 10 wherein the binding member comprises at least one human constant region.

16. The method according to claim 9 or 10 wherein said active agent is doxorubicin, oxaliplatin, taxol, tomudex, 5-Fluorouracil, Irinotecan or Cisplatin.

17. The method according to claim 16 wherein said active agent is tomudex or 5-Fluorouracil.

18. A product comprising a) a specific binding member which binds to a cell death receptor or a nucleic acid encoding said binding member and (b) a chemotherapeutic agent as a combined Preparation for the simultaneous, separate or sequential use in the treatment of cancer.

19. A pharmaceutical composition for the treatment of cancer, wherein the composition comprises a) a specific binding member which binds to a cell death receptor or a nucleic acid encoding said binding member and (b) a chemotherapeutic agent and (c) a pharmaceutically acceptable excipient, diluent or carrier.

20. (canceled)

21. The product according to claim 18 wherein the binding member is an antibody or a fragment thereof.

22. The product according to claim 18 wherein the death receptor is FAS.

23. The product according to claim 18 wherein the binding member is the anti-FAS antibody CH11.

24. The product according to claim 21 wherein the binding member comprises at least one human constant region.

25. The product according to claim 18 wherein said active agent is doxorubicin, oxaliplatin, taxol, tomudex, 5-Fluorouracil, Irinotecan or Cisplatin.

26. The product according to claim 25 wherein said active agent is tomudex or 5-Fluorouracil.

27. The pharmaceutical composition according to claim 19 wherein the cancer is one or more of colorectal, breast, ovarian, cervical, gastric, lung, liver, skin and myeloid cancer.

28. The pharmaceutical composition according to claim 19 wherein the binding member is an antibody or a fragment thereof.

29. The pharmaceutical composition according to claim 19 wherein the death receptor is FAS.

30. The pharmaceutical composition according to claim 19 wherein the binding member is the anti-FAS antibody CH11.

31. The pharmaceutical composition according to claim 28 wherein the binding member comprises at least one human constant region.

32. The pharmaceutical composition according to claim 19 wherein said active agent is doxorubicin, oxaliplatin, taxol, tomudex, 5-Fluorouracil, Irinotecan or Cisplatin.

33. The pharmaceutical composition according to claim 32 wherein said active agent is tomudex or 5-Fluorouracil.
